(19) United States
(12) Patent Application Publication
(10) Pub. No.: US 2008/0161687 A1
(43) Pub. Date: Jul. 3, 2008

(54) REPEAT BIOPSY SYSTEM

(76) Inventors: Jasjit S. Suri, Roseville, CA (US); Dinesh Kumar, Grass Valley, CA (US); Yujun Guo, Grass Valley, CA (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/750,854

(22) Filed: May 18, 2007

Related U.S. Application Data
(60) Provisional application No. 60/882,832, filed on Dec. 29, 2006.

(51) Int. Cl. A61B 8/00; A61B 10/02

(52) U.S. Cl. 600/437; 600/562

(57) ABSTRACT

Provided herein are improved biopsy systems and methods (i.e., utilities), which among other benefits, assist physicians with the selection biopsy sites based on temporally distinct images. More generally, the utilities allow for registering medical images such that information associated with a medical image obtained at a first time may be mapped to a subsequent medical image obtained at a subsequent time. This may result in improved diagnosis, better planning, better treatment delivery and/or follow-up.

### CROSS REFERENCE TO RELATED APPLICATIONS

This application claims priority under 35 U.S.C. §119 to U.S. Provisional Application No. 60/882,832 entitled “AN IMPROVED BIOPSY SYSTEM” having a filing date of Dec. 29, 2006, the contents of which are incorporated by reference herein.

### FIELD

The system and methods (i.e., utilities) disclosed herein are directed to medical imaging. More specifically the utilities are directed to registering temporally distinct images such that information from an earlier image may be transferred to a subsequent image.

### BACKGROUND

Prostate cancer is the second leading cause of death among males in USA. Accordingly, early detection and treatment of prostate cancer is important. In general, a biopsy is recommended when a patient shows high levels of PSA (Prostate Specific Antigen), which is an indicator of prostate cancer. Ultrasound guided biopsy is a commonly used method to remove suspicious tissues from the prostate for pathological tests so that malignancy can be established. There have been efforts to find optimal locations for prostate biopsies, but the selection does not guarantee that the malignancy will be detected in the first session itself. That is, the samples collected by the urologist/physician may produce a false negative if the needle does not pass through malignant tissues. If the biopsy tests are negative but there remains a presence of high PSA levels, a follow-up biopsy is usually recommended. Some studies have shown that up to 10% of negative results from first biopsy may produce positive response after follow-up biopsy.

In case of follow-up biopsy, the sites selected in previous biopsies should be excluded so that tissues from new sites can be tested. However, it is difficult for a physician to locate corresponding locations from temporally distinct images. That is, a first image that was obtained during a patient's first visit and a second image that is obtained at the time of follow-up biopsy. This is due to complexity of observing a three-dimensional space as well as due to change in shape and/or position of the prostate. The prostate shape and orientation may vary in the ultrasound images due to patient motion, motion of surrounding structures and also due to abdominal contents. It is therefore, desirable to find the correspondence between the two images so that the previous biopsy sites can be identified on the follow-up biopsy ultrasound images.

### SUMMARY

Accordingly, provided herein are improved biopsy systems and methods (i.e., utilities), which among other benefits, assist physicians with the selection biopsy sites based on temporally distinct images. More generally, the utilities allow for registering medical images such that information associated with a medical image obtained at a first time may be mapped to a subsequent medical image obtained at a subsequent time. This may result in improved diagnosis, better planning, better treatment delivery and/or follow-up. It will be noted that in addition to the overall system, that various components of the system are themselves novel.

According to a first aspect, a utility is provided for utilizing a previous image with current image in order to provide additional information for a current procedure. The method includes obtaining a first prostate image at a first time and identifying at least one point of interest in the first prostate image. The at least one point of image may then be stored with the prostate image for subsequent use. At a second time, a second prostate image is obtained. The first and second images are then registered together such that the first and second images are aligned to a common frame of reference and such that the point of interest from the first image is incorporated into the second image.

Variations and additional features exist in the above-noted utility. For instance, the point of interest associated with the first image may be, without limitation, a region of interest (e.g., a lesion or malignancy), a previous treatment location and/or a previous biopsy location. In any case, the information associated with the first prostate image may be incorporated into the second image. Further such incorporation may include the display of the point of interest on and/or within the second prostate image. Accordingly, a physician may utilize this information from the first image as displayed on the second image in order to select locations for, for example, subsequent biopsy and/or treatment of the prostate.

In one arrangement, the first and second images may be obtained during a common office visit. For instance, during a procedure in which the shape and/or position the prostate may be expected to change, it may be beneficial to register an image after the change in shape/position with an image obtained before the change in shape/position. For instance, upon inserting a needle into the prostate to obtain a biopsy sample, the prostate may move and/or rotate. If the desired biopsy locations were located in an image obtained prior to movement of the prostate, it may be desirable to transfer these desired biopsy locations into the shifted prostate image in order to obtain biopsies from the proper location(s) within the prostate. Stated otherwise, a point of interest may be located within an image prior to needle insertion and a second image may be obtained after needle insertion. These images may then be registered together in order to provide a method for guiding a needle to the desired location of interest and the shifted prostate. In other words, the image before needle insertion and image during the needle insertion need to be in same frame of reference to minimize errors due to patient movement, movement of internal anatomical structures as well as the deformation caused by the needle insertion.

In another arrangement, the first and second images are obtained at separated office visits. In this regard, a first prostate image and/or biopsy locations may be obtained during a first office visit. In a follow-up visit, the first image and locations of interest associated with the first image may be utilized with a subsequently obtained image. By registering the images together, the previous biopsy locations may be incorporated onto the current image to allow for biopsy of previous location and/or new locations. That is, a physician may decide to plan a future biopsy in a suspected region after one biopsy has already been performed and the tissue found benign in that region. The physician in such cases may want to avoid performing biopsy in the same region again to avoid unnecessary pain to patient and wastage of resources and time. This requires identification of target tissue of earlier biopsy sites in the second or newly image acquired. The newly acquired image will generally be different from the earlier image depending upon a number of factors, such as the abdominal contents, motion of the structures inside the patient, change in the shape and size of prostate as a result of the patient's response to the disease and/or treatment, and the positioning of the patient. In this regard, it is noted that the soft structures in human abdomen has a high degree of variability over time, even during the same day and unlike the chest scans (heart and lung), the variability is not periodic. The abdominal structures have different contents from day to day, and change their relative positions depending upon the patient sitting position and previous state and they may twist or rotate around other neighboring structures, e.g., for prostate, the bladder and rectum contents cause the deformation of prostate.

The utility may be utilized to perform a repeat biopsy procedure where a first three-dimensional prostate image is obtained and a biopsy is performed at one or more locations of the prostate. The biopsy locations are then mapped onto the three dimensional prostate image. At a second subsequent time, a second three-dimensional prostate image is obtained and is registered with the first prostate image. Accordingly, such registration allows for incorporating/displaying the biopsy locations of the first prostate image on the second prostate image. That is, the utility may use real-time image registration to align the image acquired from a previous scan/image to the frame of reference of the image acquired during the re-visit of patient such that the original biopsy site can be easily identified and/or avoided during a current procedure.

In order to register the first and second images, it may be necessary to segment each image. Such segmentation may be done in any appropriate manner. In one embodiment, segmentation is done utilizing an active contour method. Likewise, the images may be registered together in any appropriate manner. However, in one arrangement three-dimensional images are registered together on a two-dimensional, slice-by-slice basis. Further, such registration may begin at a center slice and extend outward. In this regard, the utility may be operative to utilize first and second processors to the segment and register the slices on either side of the middle slice. Use of the parallel processors may significantly reduce the processing time required for segmentation and registration. Accordingly, this may allow for substantially real-time registration of a current image with a previous image and thereby reduce waiting time of a patient and/or physician.

Image registration provides mapping between a pair of images such that the mapping defines the correspondence at each point in the images. For instance, the deformation caused by needle insertion has a high degree of freedom and can be better approximated by an elastic deformation rather than a rigid body or affine transformation. In one arrangement, a non-rigid inverse-consistent linear elastic image registration using b-spline basis functions is utilized. Inverse-consistency implies that the correspondence is same in both directions and there are no correspondence ambiguities. The implementation may be real-time to make it practical to use for the urologist to guide the needle to the target region of interest while image is being acquired.

In one arrangement, the images are acquired by rotating an ultrasound probe in small equal intervals (e.g., automatically) to acquire a three-dimensional image of the prostate. In such an arrangement the images may be acquired using Trans Rectal Ultra Sound (TRUS) imaging system. However, other systems (e.g., MRI, CT etc) may be utilized as well. The prostate boundaries are shown on screen and the target biopsy sites are picked by the physician. The information about the biopsy sites (e.g., points of interest) is stored electronically with the segmented image for later review as well as for comparison with any follow-up images. The comparison is made by registering the two images together and incorporating the biopsy sites from earlier an earlier image in the new image. Incorporating the points of interest from the first image onto the second image may entail displaying information associated with the points of interest on the second image. For instance, markers associated with the points of interest may be displayed on the second image.

In another aspect, an improved biopsy utility is provided in which three dimensional ultrasound image is acquired of a prostate. The prostate boundaries are extracted using a real-time segmentation and registration method that allows for guiding the needle to the target site. Based on the feedback from the pathologist and/or other reports, the urologist may want to plan more biopsy. The utility helps the urologist to make decision about the site for the next biopsy based on earlier biopsy images/scans. The sites of early biopsy locations are displayed to the physician as an overlay over the three dimensional image volume. The physician can rotate it in space, slice through it and choose the biopsy target sites by clicking on the display. The selected biopsy sites may be displayed as small markers overlaid on the three-dimensional volume. In one arrangement, the markers are colored (e.g., colored spheres). Such a display may help the physician to guide the needle to the marked biopsy site easily identifiable with a unique color. The colors may be selected such that no colors are repeated and the colors are most diverse such that they are spectrally most different from each other and from the background. The image segmentation is saved as the patient record and the biopsy sites locations are saved, along with a unique identifier tag and a unique color.

All of the aspects and utilities may include segmentation, rigid registration and elastic registration subsystems. Such a segmentation subsystem may extract the prostate from the ultrasound image in real time for use in image registration. The rigid registration subsystem may place the prostate image from a previous image in same orientation and location as a newly acquired prostate image. The elastic deformation subsystem allows for deforming the prostate images (i.e, that are in a common orientation) into a common shape. This allows mapping information in the previous image (e.g., points of interest) into the subsequent image

The registration sub-system may be trained based on a large number (e.g., 3000) of a priori datasets on which the landmark points are selected. The location and curvature statistics over large training set is used as a priori information to select the same points from each slice of a current image volume. These points are used as landmarks for rigid alignment of the images.

The elastic deformation registration subsystem may perform an inverse-consistent linear elastic image registration inside a bounding box determined by the prostate segmentations and the registration parameterization.

In a further arrangement, rather than registering entire prostate images, only portions of interest of the prostates may be registered. For instance, the elastic registration may be a memory intensive and computationally expensive process. This may be made faster by focusing only over a small region of interest (ROI) containing the prostate and performing the registration only in that small region of interest.

In another arrangement, a multi-resolution approach is applied where the image at a very small resolution is registered first and the correspondence is then used to initialize registration at a higher resolution until full resolution is reached. A multi-resolution approach has a number of advantages such as: better memory management, faster speed, better correspondence by avoiding many local minima and faster convergence.

The entire architecture may be implemented on a GPU based framework, which can make visualization and computation faster by up to factor of 30. The entire architecture may therefore be implemented in real-time.

### BRIEF DESCRIPTION OF THE DRAWINGS

FIG. 1 illustrates obtaining a prostate image;

FIGS. 2A and 2B illustrate 2-D images and a resulting 3-D image, respectively.

FIG. 3 illustrates a process for performing a biopsy procedure.

FIG. 4 illustrates a process for registering images acquired during a common examination.

FIG. 5 illustrates a process for registering images acquired during separate examinations.

FIG. 6 graphically illustrates registration of temporally distinct 2-D images.

FIG. 7 graphically illustrates registration of temporally distinct 3-D images.

FIG. 8 illustrates landmark points in a training image.

FIG. 9 illustrates a process for 3-D prostate segmentation sing parallel processing.

FIGS. 10A-D illustrate a first prostate image, a second prostate image, overlaid prostate images prior to registration and overlaid prostate images after registration, respectively.

FIG. 11 illustrates a process for rigid and elastic registration of first and second images.

FIG. 12 illustrates a process for rigid registration.

FIG. 13 illustrates a process for elastic registration.

FIG. 14 illustrates a color scheme for marking points of interest with unique colors.

### DETAILED DESCRIPTION

Reference will now be made to the accompanying drawings, which assist in illustrating the various pertinent features of the present disclosure. Although the present disclosure is described primarily in conjunction with transrectal ultrasound imaging for prostate imaging, it should be expressly understood that aspects of the present invention may be applicable to other medical imaging applications. In this regard, the following description is presented for purposes of illustration and description.

Disclosed herein are systems and methods (i.e, utilities) that facilitate, inter alia, obtaining biopsy samples from a tissue areas of interest when temporally different images are used for biopsy sample guidance. As may be appreciated, on the recommendation of a primary care physician, a prostate biopsy may be performed by a urologist. The recommendation is usually made on the basis of high Prostate-Specific Antigen (PSA) and Prostatic Acid Phosphatase (PAP) in the blood. The high levels of PSA and PAP may also occur naturally or due to other reasons, so primary care usually recommends a patient to a urologist for more tests. The urologist looks at the ultrasound scan of the prostate and identifies a suspicious region for further tests. The urologist then proceeds to extract tissue from this biopsy site(s).

If the biopsy samples are negative, but, the PSA levels remain high there may be a need for follow-up scan or a follow-up biopsy, which may occur anywhere from a few months to more than a year after the initial biopsy procedure. In general, the data from the previous scan cannot be used directly to plan new biopsy sites or even to monitor old sites. The main reason for this is the change in shape of prostate over the period of time. The prostate may have expanded as a result of some growing malignancy, or may have contracted in response to some illness or medication, or there might have been some shape changes due to variety of reasons. It is almost impossible for the urologist to exactly identify each point from previous biopsy site in the new image. The presented utilities solve this problem and other problems by aligning a previous dataset of an image with a new image. That is, the utility registers a segmented prostate image from earlier scan to a new segmented prostate image. This provides the output in the form of a transformation of co-ordinates from previous image to the current image. Accordingly, points of interest, such as previous biopsy locations, are also loaded and displayed in the co-ordinates of the new image. This information helps urologist in deciding the region, or sites for the new biopsy

Generally, the utilities incorporate a medical imaging registration system that allows a first medical image of a tissue area of interest that acquired at a first time to be registered with a second medical image of the tissue are of interest that is acquired at a second subsequent time. Accordingly, once the temporally distinct images are registered, information associated with the first image may be transferred to the subsequent image for treatment purposes. As noted, such registration of images may be beneficial in instances where considerable time has elapsed between the images (e.g., separate office visits). However, such registration may also be beneficial during a procedure in which the shape and/or location of an imaged object changes during the procedure. For instance, during biopsy sampling of a prostate, the insertion of a needle into the prostate may deform the prostate, which may cause the needle to miss a desired target region within the prostate.

The following description is divided into a number of sections. In the Introduction section, the acquisition of medical images is first described. Thereafter, an overview of instances where application of registration of temporally distinct images is desirable is presented. Finally, the last section describes the implementation of various components that allow for segmenting as well as registering temporally distinct images.

### Image Acquisition

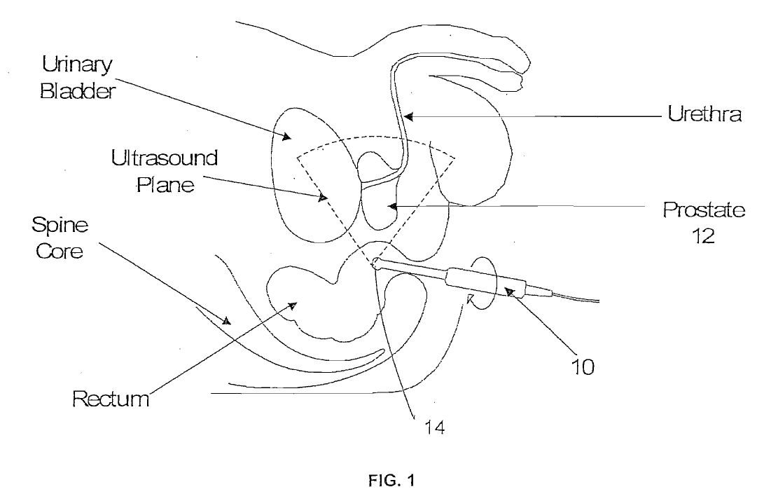

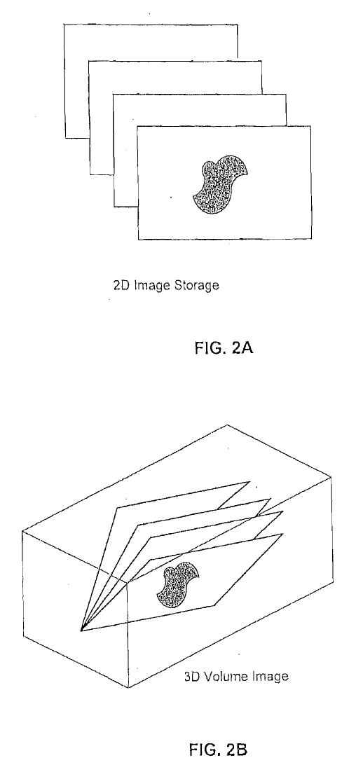

FIG. 1 illustrates a transrectal ultrasound probe being utilized to obtain a plurality of two-dimensional ultrasound images of the prostate 12. As shown, the probe 10 may be operative to automatically scan an area of interest. In such an arrangement, a user may rotate the acquisition end 14 of the ultrasound probe 10 over an area of interest. Accordingly, the probe 10 may acquire plurality of individual images while being rotated over the area of interest. See FIGS. 2A-B. Each of these individual images may be represented as a two-dimensional image. See FIG. 2A. Initially, such images may be in a polar coordinate system. In such an instance, it may be beneficial for processing to translate these images into a rectangular coordinate system. In any case, the two-dimensional images may be combined to generate a 3-D image. See FIG. 2B.

As shown in FIG. 1, the ultrasound probe 10 is a side fire probe that generates ultrasound waves out of a side surface. However, it will be appreciated that an end fire scan probe may be utilized as well. In any arrangement, the probe 10 may also include a biopsy gun (not shown) that may be attached to the probe. Such a biopsy gun may include a spring driven needle that is operative to obtain a core from desired area within the prostate. In this regard, it may be desirable to generate an image of the prostate 12 while the probe 10 remains positioned relative to the prostate. If there is little or no movement between acquisition of the images and generation of the 3D image, the biopsy gun may be positioned to obtain a biopsy (or perform other procedures) of an area of interest within the prostate 12. However, manual manipulation of the probe 10 often results in relative movement between the probe and the prostate 12 between subsequent images and/or as a biopsy device is guided toward an area of interest.

Accordingly, for imaging is desirable that relative movement (e.g., wobble) between the probe 10 and the prostrate 12 be minimized (i.e., other than rotational movement of the probe about a fixed axis for image acquisition). Further, it is often desirable that the probe remains fixed relative to the prostrate 12 during biopsy or other treatment procedures such that desired tissue locations may be accurately targeted. To achieve such fixed positioning of the probe, it is often desirable to interface the probe 10 with a positioning device that maintains the probe 10 in a fixed relative position to the prostate.

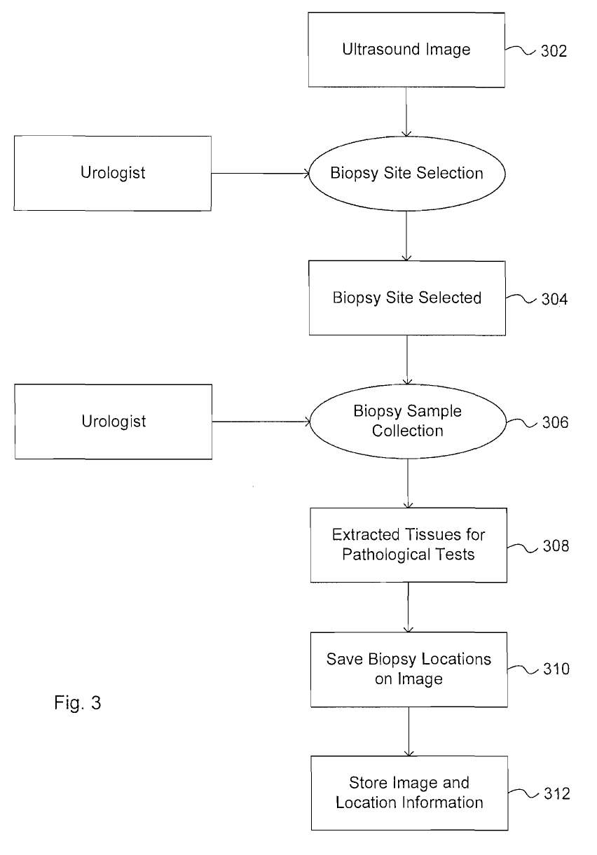

FIG. 3 illustrates the acquisition of an ultrasound image and the identification of biopsy locations, biopsy sample collection and the recording of biopsy sample locations with the three-dimensional image. Initially, an ultrasound image is acquired 302. Such acquisition may be preformed using a TRUS as discussed above. Once the image is acquired, biopsy sites are selected 304. Once the biopsy sites are selected, biopsy sample collection is preformed 306 such that extracted tissues for pathological tests may be provided 308. In conjunction with the biopsy sample collection and extraction, the system may utilize a device for holding the biopsy device such that it is maintained in a known positional relationship to the prostate of the patient and hence an image of the prostate. For instance, such a device is disclosed in co-pending U.S. application Ser. No. 11/691,150 entitled UNIVERSAL ULTRASOUND HOLDER AND ROTATION DEVICE, having a filing date to Mar. 26, 2007 the entire contents of which are incorporated by reference herein. As the position of the biopsy needle may be know in relation to the ultrasound image, the locations within the prostate from which biopsy samples are extracted may be saved 310 into the ultrasound image. Once all biopsy locations are saved into the image, the composite image including the information associated with the biopsy locations may be stored 312 for future use.

### Applications

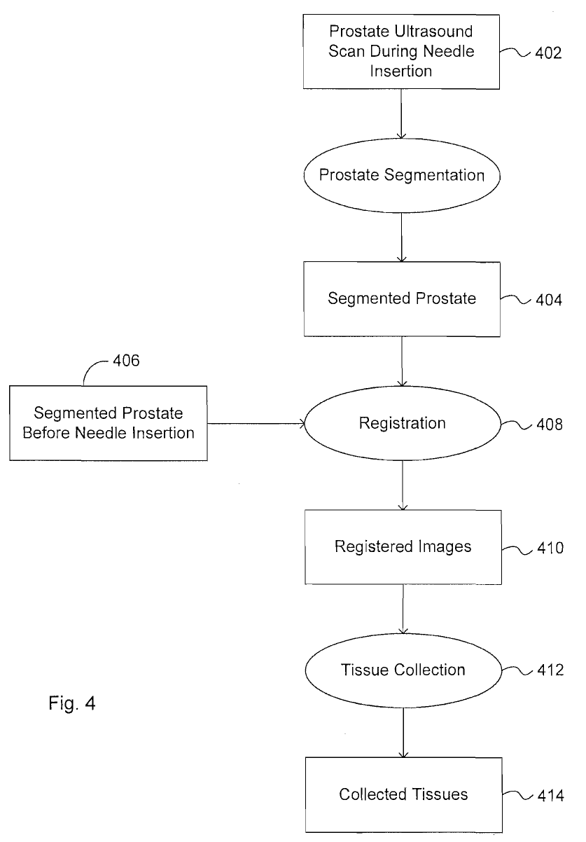

FIG. 4 provides an overview of use of a previous three-dimensional volume (e.g., ultrasound image) with a new (e.g., current) three-dimensional image in order to perform one or more procedures on the prostate based on a combination of the images. More specifically, FIG. 4 provides a processes flow diagram for tissue sample collection wherein an initial ultrasound image obtained during a biopsy procedure is utilized to correct for changes in the shape of the prostate during needle insertion so that tissue may be collected from desired locations within the prostate. In this regard, it will be appreciated that insertion of the needle into the prostate may change the shape thereof. Accordingly, regions selected within the prostate for biopsy may move upon needle insertion. By registering the image of the prostate after needle insertion with an image of the prostate prior to needle insertion, corrections may be made to the location of the targeted locations within the prostrate and hence the needle trajectory such that samples are obtained from desired target locations. Accordingly, the process includes obtain an ultrasound scan during needle insertion 402 and segmenting the image 404. The method further includes obtaining a segmented prostate image that is obtained prior to needle insertion 406. These before and after needle insertion segmented images are then registered together 408 in order to generate one or more registered images 410. At this time, differences between the images may be identified such that corrections to the needle trajectory may be made in order to improve tissue collection 412. Accordingly, the collected tissue 414 may be provided for pathological testing.

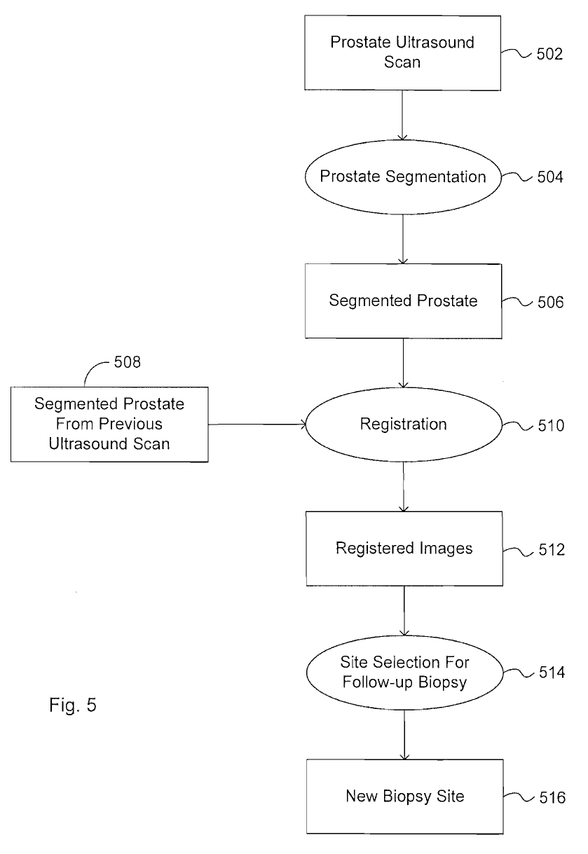

FIG. 5 illustrates a process flow diagram for the selection of biopsy sites based on previous biopsy locations. In this arrangement, it will be appreciated that a previous biopsy may have been negative. However, due to one or more factors such as, for example, elevated PSA levels it may be desirable to perform a follow-up biopsy. However, it may also be desirable to perform biopsy at new locations within the prostate. Accordingly, a previous prostate image that includes biopsy locations may be retrieved for use with the current image. Specifically, a current prostate ultrasound image is obtained 502. This current ultrasound image may then be segmented 504 to generate a segmented prostate image 506. A segmented prostate image from a previous ultrasound image scan that includes one or more previous biopsy locations may then be retrieved 508. These images may be registered together 510 in order to generated register images 512. Once the images are registered together, site selection may be identified for new/follow-up biopsy 514. Samples may then be obtained from the new biopsy sites 516. Likewise, these new biopsy locations may be saved within the current image and may be utilized at a further subsequent procedure.

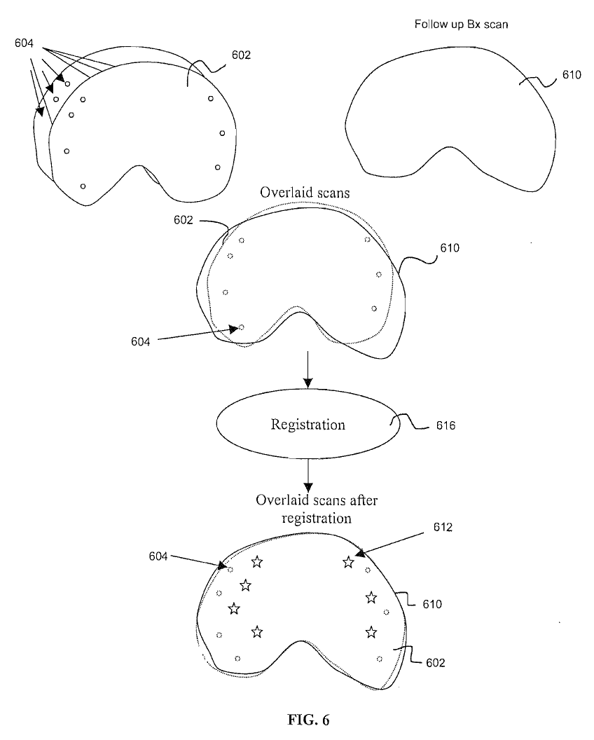

This process is graphically illustrated in FIG. 6. In this regard, FIG. 6 may be indicative of a graphical interface that a physician may utilize while performing a follow-up biopsy procedure. As shown, a previously obtained image 602 that includes a plurality of previous biopsy sites 604 mapped to the image is obtained. Likewise, a follow-up image 610 of the prostate is obtained. This follow-up image 610 is segmented so that it may be registered with the previous image, as will be discussed herein. As shown, the shape of the prostate from the new scan 610 and the prostate 602 of the previous scan do not match in shape when overlaid. That is, when the scans are overlaid the boundaries of the prostate images do not align. Accordingly, a registration process is utilized to align the boundaries of the images 602, 610. This registration 616 may be preformed in any appropriate manner, and one specific methodology is discussed herein. Once the images 604, 610 are registered and overlaid the previous biopsy locations 604 may be illustrated on the current biopsy image 610. Accordingly, new target sites 612 may be selected based on the location of the previous biopsy sites 604.

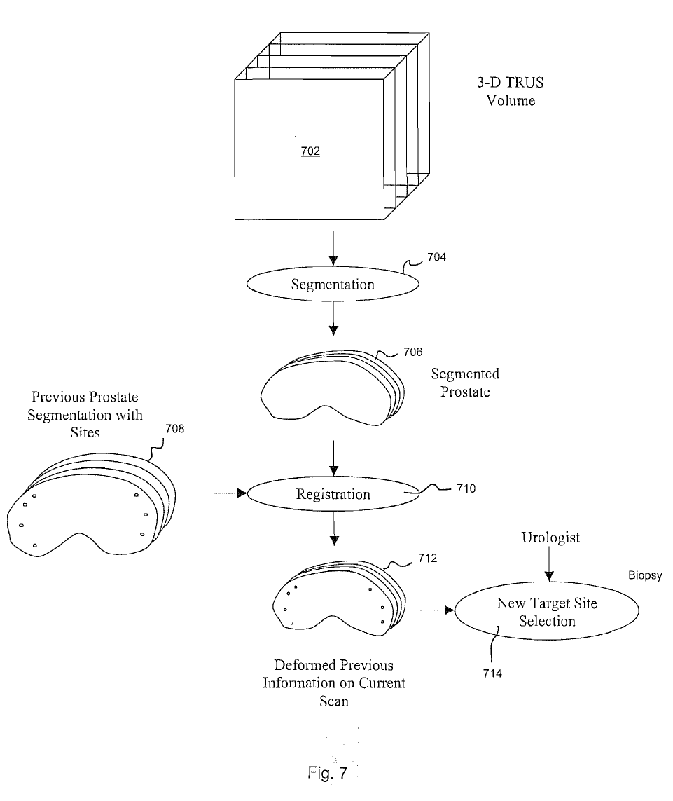

As illustrated in FIG. 6 a single prostate image is overlaid on a previous single image for instance, a single slice of a three-dimensional scan is overlaid onto a previous single slice of a three-dimensional scan. It will be appreciated, that the same process may be utilized on a three-dimensional volume as is illustrated in FIG. 7. As shown, a three-dimensional volume 702 may be obtained and segmented 704 to provide a plurality of segmented prostate slices 706. Accordingly, a plurality of previous prostate segmented slices including previous biopsy locations may be obtained 708. This plurality of slices may then be registered together 710 in order to provide a plurality of segmented registered slices that may define a 3D registered volume. Accordingly, physicians may utilize the registered slices/volume for a new target site selection. In this regard, the physician may select individual slices from within the three-dimensional registered volume and/or create new views of the volume in order to identify new locations of interest for biopsy.

### Components

1. Prostate Segmentation:

Prostate segmentation is an important part of the proposed system. Manual segmentation of prostate from noisy ultrasound images is difficult in 2-D images and is typically prohibitively difficult in 3-D images. Therefore, a robust and fast segmentation method is needed. The current system utilizes a modified Gradient Vector Field based segmentation method for segmentation of prostate from the ultrasound image. The segmentation method requires an initial contour which is fine-tuned to fit the prostate boundaries more precisely by deforming through an elastic transformation. One such segmentation process that allows for identifying a contour is provided in U.S. patent application Ser. No. 11/615,596, entitled “Object Recognition System for Medical Imaging,” filed on Dec. 22, 2006, the entire contents of which are incorporated herein by reference.

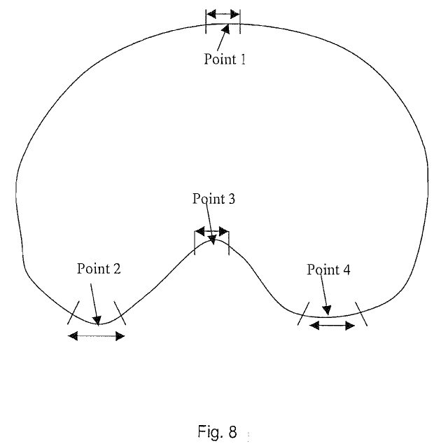

A large training dataset is used for computing an initial contour, which is used as an initialization for the segmentation. In this regard, the system also has a priori information from a large set of training data, for example, a training set consisting of about 3000 datasets. Each dataset represents a 2-dimensional image slice of prostate and the segmented boundaries can be seen as shown in FIG. 8. Four points are identified on the figure and the curvature statistics is computed for of these points over the entire set. This data is available internally to the presented system. When a urologist runs a scan, then these points are automatically selected using the curvature information from the underlying a priori information. These points depend on anatomy and hence provide a better guide for alignment than just the boundaries without correspondence.

The segmentation of prostates in the training dataset is done in 2-D, i.e. one slice at a time. This may be performed in a semi-automatic method, where user picks a small number of points at the prostate boundaries in the middle slice (i.e., 2-D slice) of the 3-D ultrasound image. The boundary points of this 2-D slice are approximated using an ellipsoid, which is then deformed using active contours, as discussed later in this section. The contour converges to prostate boundaries and provide the segmentation of the boundaries in the middle slice. The process is repeated for a large number of training datasets. The average contour (i.e., near contour) over the training datasets is used as the initialization. The average contour is computed by computing average co-ordinates over sampled contours corresponding to the training samples.

The concept of an active contour model or snakes is discussed in U.S. patent application Ser. No. 11/615,596, incorporated by reference above. The method takes as input an initial contour and deforms the contour in normal direction through a driving force derived from image gradient. This method is used to compute 2D segmentation on each slice of the 3D prostate segmentation to provide a 3D segmentation of the prostate.

In this method, static external force field v(x,y) is defined as v(x,y)=[u(x,y),v(x,y)] that minimizes the energy functional:

ε=∫∫μ(uₓ²+u_{y}²+vₓ²+v_{y}²)+|∇f|²|v−∇f|²dxdy | (1)

where f(x,y) is the edge map derived from image I(x,y) having the property that it is larger near the image edges. This energy equation produces the effect of keeping v nearly equal to the gradient of the edge map f(x,y) when it is large, and forcing the field to be slowly-varying in homogeneous area. Regularization parameter (m) governs the tradeoff between the first and second term, and should be set according to the noise presented in the image. The system needs larger m, for larger noise. The contour deformation field is found by solving the following Euler equations using the calculus of variations

μ∇²u−(u−fₓ)(fₓ²+f_{y}²)=0μ∇²v−(v−f_{y})(fₓ²+f_{y}²)=0 (2)

where is the Laplacian operator and the edge map f is defined as

f(x,y)=|∇[G_{σ}(x,y)*I(x,y)]² (3)

where G_{σ}(x,y) is the Gaussian kernel with given σ, I(x, y) was the given image, and * is the convolution operator.

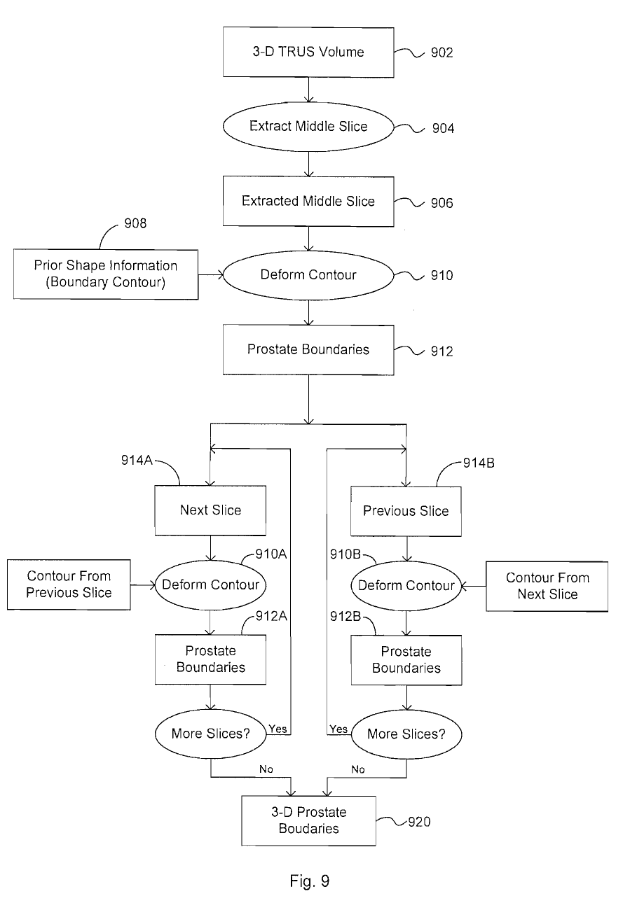

The use of 2-D segmentation to identify 3-D prostate boundaries is set forth in FIG. 9. Initially, a 3D image volume is acquired 902 from the patient. A middle slice of the prostate is identified 904 and extracted 906. The system automatically initializes the contour in the middle slice using the average contour obtained 908 from the training datasets. The contour is then deformed 910 to fit the prostate boundaries of the middle slice using method described above. The contour obtained from the middle slice is propagated to the adjacent slices before and after the middle slice as an initial contour in those slices. These contours are again fine-tuned using the same method and the process is repeated till all the slices are traversed and the method yields a 3D prostate segmentation.

That is, the prostate segmentation system is implemented to take advantage of dual-processors such that once the middle slice is segmented, the segmentation is propagated 914A, 914B in two directions simultaneously: to the slices before middle slice and slices after the middle slice. Both processes are assigned to different processors as there is no interaction between the two steps which makes the segmentation faster. At the same time, the landmark points are extracted using a-priori information, thus the entire process is highly parallel once the middle slide is segmented, and may be easily distributed over different processors. This makes the implementation faster and real-time. Once all prostate boundaries are identified for all slices, a 3D boundary is generated 920.

2. Image Registration:

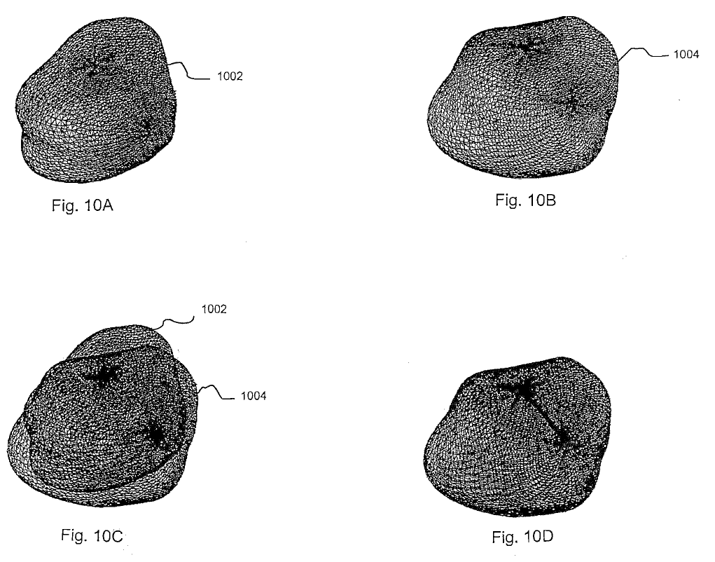

In medical imaging, image registration is used to find a deformation between a pair or group of similar anatomical objects such that a point-to-point correspondence is established between the images being registered. The correspondence means that any tissue or structure identified in one image can be deformed back and forth between the two images using the deformation provided by the registration. FIGS. 10A and 10B illustrate first and second prostate images 1002 and 1004, for example, as may be rendered on an output device of physician. These images may be from a common patient and may be obtained at first and second temporally distinct times. Though similar, the images 1002, 1004 are not aligned as shown by an exemplary overlay of the images prior to registration (e.g., rigid and/or elastic registration). See FIG. 10C. In order to effectively align the images 1002, 1004 to allow transfer of data (e.g., prior biopsy locations) from one of the images to the other, the images must be aligned to a common reference frame and then the prior image (e.g., 1002) may be deformed to match the shape of the newly acquired image (e.g., 1004). In this regard, corresponding structures or landmarks of the images may be aligned to position the images in a common reference frame.

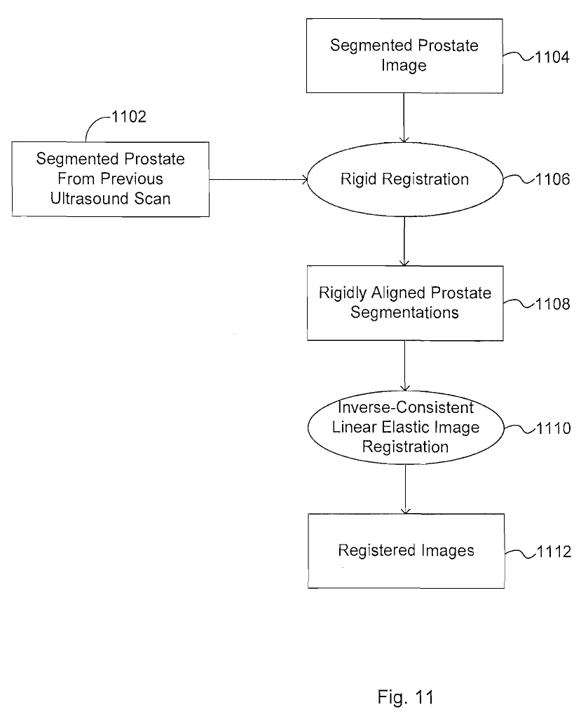

The correspondence is defined everywhere in the object in the continuum. There are various models of registration depending upon the objective and in general, for anatomical objects, a non-rigid elastic deformation model is used. To avoid correspondence ambiguities associated with most unidirectional registration methods, an inverse-consistent image registration method is used. The objective functions used for registration dictate that the initial overlap of the object is good; this is ensured by performing a rigid alignment before performing the elastic registration. This is illustrated in FIG. 11, where first and second segmented images 1102 and 1104 are rigidly registered 1106 to produce rigidly aligned prostate segmentations 1108. The rigid alignment provides a good starting point for the elastic registration 1110, which finds the closest local minima during its search for optimized solution (i.e. registration) 1112. The rigid registration and elastic registration methods are explained in more details as follows:

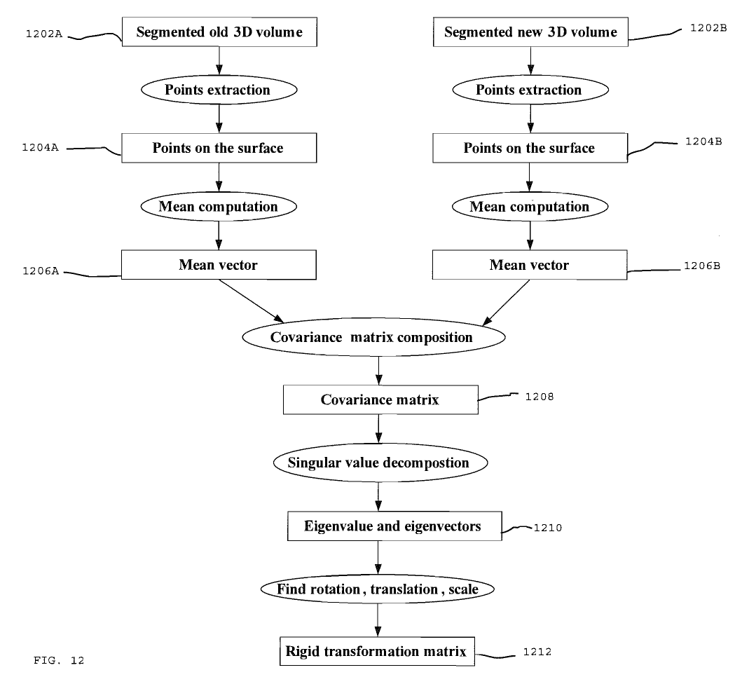

A good initial rigid alignment is needed as a good starting point for the elastic registration method to produce a valid result. FIG. 12 illustrates a rigid registration method. For performing the rigid alignment, segmented images 1202A-B are provided for the previous and newly acquired images. Boundary points are extracted 1204A-B (i.e, in parallel processes in the present embodiment) from the segmentations. The points are used to generate mean vectors 1206A-B for the images. These vectors are utilized to define a covariance matrix 1208 that is solved to provide eigenvalues/eigenvectors 1210 that are used together to generate rotation and translation parameters that may be applied to one of the images (e.g., the previous image) to align it with the newly acquired image. That is, a rigid registration matrix is computed 1212 using the vector information. The rigid registration matrix provides the following transformation in the co-ordinate system:

$$( \begin{matrix}{x}^{′} \\ {y}^{′} \\ {z}^{′} \\ 1\end{matrix} ) = [ \begin{matrix}{a}_{xx} & {a}_{xy} & {a}_{xz} & Δ x \\ {a}_{yx} & {a}_{yy} & {a}_{yz} & Δ y \\ {a}_{zx} & {a}_{zy} & {a}_{zz} & Δ z \\ 0 & 0 & 0 & 1\end{matrix} ] ( \begin{matrix}x \\ y \\ z \\ 1\end{matrix} ) \qquad ( 4 )$$

where, x′, y′ and z′ are the deformed co-ordinates after application of rigid registration matrix on the original co-ordinates x, y and z. The rigid alignment only allows translation and rotation, and therefore does not include scaling or shearing or any nonlinear operations. Hence, the determinant of the transformation matrix is always one. The rigid alignment orients the previous image to the location of the current/newly acquired image such that the translational and rotational differences are minimized. After rigid alignment, the next step is to perform the non-rigid registration (e.g., elastic deformation) of the rigidly aligned images.

The rigid alignment improves the relative overlap of the two regions to be registered together. For computing inverse consistent linear elastic image registration, the aim is to perform registration only in a small bounding box containing the prostate. However, it will be appreciated that if a smaller region of interest is desired, the bounding box may be sized to only extend about the region of interest. In one embodiment, a B-spline basis is used for the registration and defining the bounding box based on the extents of the object and the knot spacing of B-splines. The bounding box is initially obtained by computing the union of the prostate segmentations from the two images and computing the extents of the union. The bounding box is then extended to contain the knot points corresponding to the B-splines supporting the region inside boundary box. This padding is required not only for better approximation using B-spline basis functions, but also to provide a cushion for deformation of the object. A multi-criterion objective function approach if followed for the registration inside the bounding box. Spatially defined B-spline basis functions are used for parameterization. For a pair of images I1 and I2, the registration problem is described as the problem of finding transformations h1,2 from image I1 to image I2 and h2,1 from image I2 to image I1, respectively, that minimize the following cost function:

$$C = σ ( {\int }_{Ω} {\vert {I}_{1} ( {h}_{1 , 2} ( x ) ) - {I}_{2} ( x ) \vert}^{2} d x + {\int }_{Ω} {\vert {I}_{2} ( {h}_{2 , 1} ( x ) ) - {I}_{1} ( x ) \vert}^{2} d x ) + ρ ( {\int }_{Ω} {‖ L ( {u}_{1 , 2} ( x ) ) ‖}^{2} d x + {\int }_{Ω} {‖ L ( {u}_{2 , 1} ( x ) ) ‖}^{2} d x ) + χ ( {\int }_{Ω} {‖ {h}_{1 , 2} ( x ) - {h}_{2 , 1}^{- 1} ( x ) ‖}^{2} d x + {\int }_{Ω} {‖ {h}_{2 , 1} ( x ) - {h}_{1 , 2}^{- 1} ( x ) ‖}^{2} d x ) \qquad ( 5 )$$

where, I1(x) and I2(x) represent the intensity of image at location x, represents the domain of the image. hi,j(x)=x+ui,j(x) represents the transformation from image Ii to image Ij and u(x) represents the displacement field. L is a differential operator and the second term in Eq. (2) represents an energy function σ, ρ, and χ are weights to adjust relative importance of the cost function.

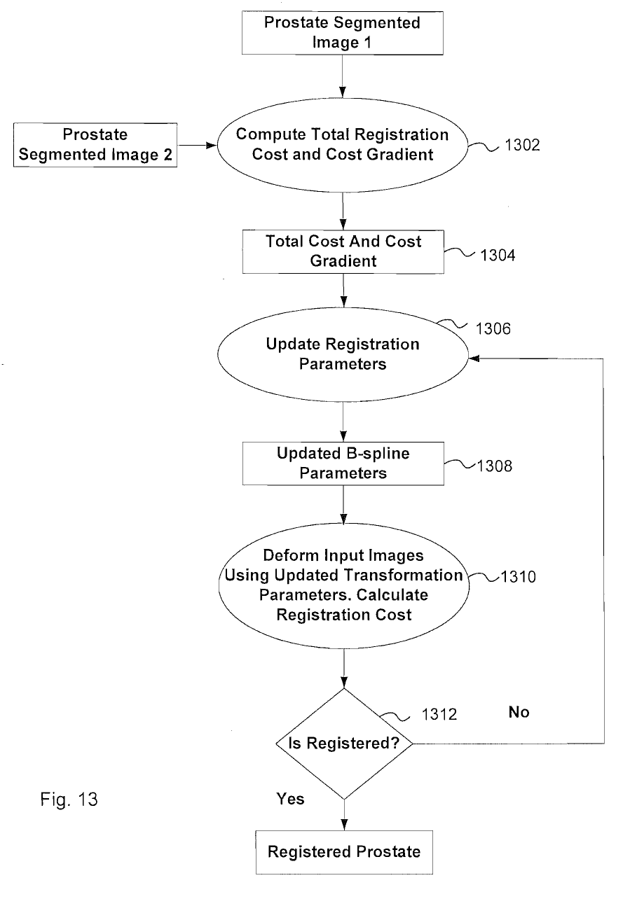

In equation (2), the first term represents the symmetric squared intensity cost function and represents the integration of squared intensity difference between deformed reference image and the target image in both directions. The second term represents the energy regularization cost term and penalizes high derivatives of u(x). L is represented as Laplacian operator mathematically given as: L=Ÿ². The third and last term represents the inverse consistency cost function, which penalizes differences between transformation in one direction and inverse of transformation in opposite direction. The total cost is computed 1302 as a first step in registration as shown in FIG. 13. The total coast and the cost gradient 1304 are then solved in an iterative process 1316-1312.

The optimization problem posed in Eq. (5) is solved by using a B-spline parameterization 1308. B-splines are chosen due to ease of computation, good approximation properties and their local support. It is also easier to incorporate landmarks in the cost term if a spatial basis function is used. The above optimization problem is solved by solving for B-spline coefficients ci's, such that

$$h ( x ) = x + {\sum }_{i} {c}_{i} {β}_{i} ( x ) \qquad ( 6 )$$

where, bi(x) represents the value of B-spline at location x, originating at index i. In the present registration method, cubic B-splines are used. A gradient descent scheme is implemented based on the above parameterization. The total gradient cost is calculated 1310 with respect to the transformation parameters in each iteration. The transformation parameters are updated 1306 using the gradient descent update rule. Images are deformed 1310 into shape of one another using the updated correspondence and the cost function and gradient costs are calculated until convergence/registration 1312.

The registration may be performed hierarchically using a multi-resolution strategy in both, spatial domain and in domain of basis functions. The registration is performed at ¼th, ½ and full resolution using knot spacing of 8, 16 and 32. In addition to being faster, the multi-resolution strategy helps in improving the registration by matching global structures at lowest resolution and then matching local structures as the resolution is refined.

3. Selection of Biopsy Sites:

The urologist is provided with the segmentation of prostate overlaid in color over a 3-D volume, which he can browse through. The urologist picks the biopsy targets by clicking on the points in the segmented image. The biopsy sites may be displayed as small spheres overlaid on the 3-D ultrasound volume and the centers of these sites are stored on the computer along with other patient data. The biopsy sites may be stored such that each site has a unique color tag and its co-ordinates for identification at a later date. The colors are assigned such that there is one and only one assignment of color to each biopsy point. Moreover, the color has to be bright enough to be able to be distinguished from gray background.

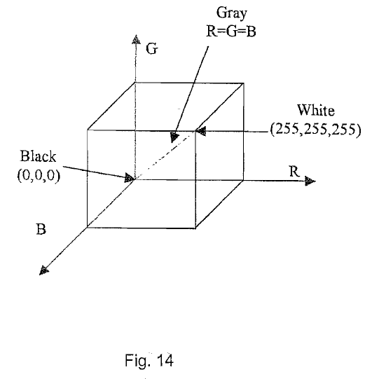

In digital imaging, a color is usually formed by combination of three primary colors: red, blue and green. Hence, every color on a computer screen can be represented using intensities of these three components. In a 24-bit color scheme (1.6 million colors on computer screen), there are 256 intensity levels each, corresponding to red, green and blue colors. The color space can therefore be represented as a 3D space with R (red), G(green) and B(blue) colors as its three axis, with the values ranging from 0 to 255, as shown in FIG. 14. Each color on screen can then be represented by a point in this color space. For example, pure black color corresponds to co-ordinates (0,0,0) as each of its R-, G-, and B-component has intensity of zero. Likewise, any intensity where values of R-, G- and B- are equal to each other, represents a gray color (diagonal line in the cube). The white color has co-ordinate location (255, 255, 255) and so on. The colors on the diagonally opposite corners of the cube are said to be furthest from each other. We use Euclidean metric defined on a metric space for computing the “distance” between the colors. By distance, we mean the difference between colors.

Using this color space, we create a table of colors that are furthest away and store them in a look up table. The main criterion is that any color used is prohibited for future use. The colors are picked as follows: 1. All gray levels are picked as the set of used, i.e. prohibited colors. 2. The colors should be bright, so we choose colors that have intensity values>100 for all colors to be selected. 3. The three primary colors with intensity values of (255, 128, 128), (128, 255, 128) and (128, 128, 255) are selected as the first three colors on a look up table. Using all the gray scale colors and the three primary colors, the next colors are picked such that they have highest average Euclidean distance from all the previously selected points. In case of ambiguity owing to symmetry of the color space, the color having highest red intensity will be selected, with green getting second priority and blue getting the least priority. Although this resolution of ambiguity appears arbitrary, it provides a robust and reproducible system for color selection. A look-up table is thus constructed with millions of colors, with the initial colors having high average Euclidean distance among them and as the number of colors increase, the average Euclidean distance also decreases. In a typical biopsy, a urologist does not generally go beyond a total of 90 biopsy sites. Although first 90 colors from the LUT can be picked that are very different from each other, the system does not limit number of sites to only 90 and any number of unique colors can be picked to assign to each new biopsy site.

As explained above, the color LUT is pre-computed and available as part of the system. During the first visit of patient, the A color scheme with 256 colors assigned each to red, green and blue channels respectively, can have 256×256×256 different colors and the colors are picked such that they are spectrally furthest from each other while coloring all the biopsy sites. All the biopsy sites are thus uniquely colored. The urologist collects the samples from each of the sites selected on computer by guiding the needle through the probe and into the prostate. This is visualized in real-time on the computer and the real-time images are available to the urologist. The segmented image and the site locations are stored in a database for any later reference.

If there is a need for follow-up scan or a follow-up biopsy, which may occur anywhere from a few months to more than a year, then the segmented data along with the sites data is retrieved from the database. The patient is again scanned and segmentation is done in real time. In general, the data from the previous scan can not be used directly to plan new biopsy sites or even to monitor the old sites. The main reason for this is the change in shape of prostate over the period of time. The prostate may have expanded as a result of some growing malignancy, or may have contracted in response to some illness or medication, or there might have been some shape changes due to variety of reasons. It is almost impossible for the urologist to exactly identify each point from previous biopsy site in the new scan. The presented system solves this problem by aligning the previous dataset with the new image. The system registers the segmented prostate from earlier scan to the new segmented prostate. This provides the output in the form of a transformation of co-ordinates from previous scan to the prostate scan being acquired. The sites are also loaded and displayed in the co-ordinates of the new image. This information helps urologist in deciding the region, or sites for the new biopsy.

The presented system is flexible enough to allow any amount of offline information to be added to the system for follow-up procedure. Such additional information may be helpful in making the system identify locations for new biopsy based on, for example historical/statistical data. For instance atlas information associated with statistically probable regions of cancer based on one or more demographic factors may be used to suggest new biopsy targets. One exemplary atlas is provided in co-pending U.S. application Ser. No. 11/740,808, entitled “IMPROVED SYSTEM AND METHOD FOR 3D BIOPSY”, having a filing date of Apr. 26, 2007, the contents of which are incorporated by reference herein. The atlas is a probability map derived from histology data over a large number of patients and has information about the probability of occurrence of cancer in a specified region. The sites with higher probability are displayed to the urologist along with the sites from previous biopsy, thus providing an intelligent guideline for the doctor to perform the procedure. Likewise, offline MRI fusion data can also be incorporated to help the urologist identify the structures in finer detail such that task of picking the biopsy targets is made simple. The presented system is thus well suited and can be expanded to incorporate any amount and any type of input information that can help in making the diagnosis and treatment better.

The system provides some additional advantages that should be noted. For instance, it is noted that the system is an integrated system which scans 3D trans-rectal ultrasound images of prostate and provides real-time display to the urologist while scanning and while performing the biopsy. The real-time registration also ensures that any shape changes occurring from insertion of needle for performing biopsy are corrected such that the biopsy sites selected by the urologist on the computer display are accurately identified during the biopsy.

Another advantage of the implementation of the segmentation technique is that it is made fast by parallel implementation, such that there are multiple slices being segmented at the same time in different threads or on different processors. Hence, the segmentation implementation is accurate, robust and fast.

The system can also provides a urologist with an interactive display, where the doctor can rotate, zoom or slice in the 3-dimensional image displayed on the screen with segmentation overlaid on the image volume. The system allows the urologist to keep track of biopsy sites in real-time by allowing him to select the sites on the computer display by clicking of computer mouse. The biopsy sites are clearly displayed and each site has its own unique id and unique color. The site movement is also tracked through image registration, which is a clear advantage over all existing systems.

The colors of biopsy sites may be selected such that the colors are unique and they are spectrally most different from each other. A color scheme is implemented such that most different colors are sequentially arranged in a pre-computed look up table such that any number of sites can be selected and assigned a unique color, making the sites easy to identify and easy to track on a later date. Another advantage of having a unique color scheme and unique tags for biopsy sites is that the patient information is properly stored on computer or in an archive for easy future reference. The information contains the segmentation and the biopsy site information including the co-ordinates, unique tags and unique colors with the patient data.

The system is flexible so that it can be extended to include all the off-line developed tools and additional information, such as atlas containing probability maps and MRI structural images to provide an intelligent tool to aid urologist for cases requiring follow-up biopsy. Another advantage is that the urologist may be provided with all the information from a previous image and from a probabilistic atlas, which is displayed to him on a computer display. The information from previous image together with a-priori information driven segmentation and registration provide a robust and accurate system to aid the urologist.

Registration is done only in a small field of view containing the prostate and is therefore fast. Registration is made faster and better using a multi-resolution strategy, which not only makes the registration run faster, but also improves registration by minimizing global differences first and then hierarchically minimizing more local differences.

1. A method for use with prostate imaging, comprising:
obtaining a first prostate image at a first time and identifying at least one point of interest within the first prostate image;
storing the first prostate image with information associated with the at least one point of interest;
obtaining a second prostate image at a second time;
registering the first and second images, wherein the information associated with the at least one point of interest is incorporated into the second image.

2. The method of claim 1, wherein the first and second images are obtained during a common examination.

3. The method of claim 2, wherein the point of interest is a desired biopsy location and the first image is acquired prior to a needle insertion and the second image is acquired after the needle is at least partially inserted into a prostate of a patient.

4. The method of claim 3, wherein a local deformation of the prostate following the needle insertion is approximated by an inverse-consistent image registration method.

5. The method of claim 1, wherein the first and second images are obtained during first and second separate examinations, respectively.

6. The method of claim 5, wherein the at least one point of interest comprise biopsy locations associated with prostate biopsies taken during the first examination.

7. The method of claim 6, further comprising:
identifying biopsy sample locations during the second examination based on the biopsy locations of the first image.

8. The method of claim 1, wherein incorporating the at least one point of interest into the second image comprises:
graphically representing the point of interest on or within the second image.

9. The method of claim 8, wherein graphically representing comprises displaying a colored marker on or within the second image, wherein each point of interest has a unique colored marker.

10. The method of claim 1, wherein the first and second images are segmented prior to the registering.

11. The method of claim 1, wherein the segmentation is three dimensional segmentation.

12. The method of claim 11, wherein the three dimensional segmentation is performed on two dimensional slices of a three dimensional volume.

13. The method of claim 12, wherein segmentation begins from a mid slice of the three dimensional value and then propagates it on either side to find full prostate segmentation in the three-dimensional image.

14. The method of claim 13, wherein the segmentation is implemented on at least two individual processors simultaneously.

15. The method of claim 10, wherein the segmentation is based on active contours.

16. The method of claim 10, wherein resulting image segmentations are saved as a patient record and the points of interest locations of each segmentation image are saved with the image segmentation.

17. The method of claim 1, wherein registering comprises performing a rigid registration that places the first image in a common orientation and location as the second image.

18. The method of claim 17, wherein registering further comprises performing a non-rigid registration to fit the first image to the second image after the images are in the common orientation.

19. The method of claim 18, wherein the points of interest of the first image are overlaid onto the second image.

20. The method of claim 18, wherein performing the non-rigid registration comprises performing an inverse-consistent linear elastic image registration inside a bounding box determined by the prostate segmentations and the registration parameterization.

21. The method of claim 1, wherein registering is only performed for a region of interest of the images, wherein the region of interest is a subset of the entire images.

22. The method of claim 1, wherein registration is performed using a multivariable resolution.

23. The method of claim 1, further comprising:
incorporating statistical information into the second image.

24. A method for use with prostate imaging, comprising:
obtaining a first 3D prostate image of a prostate of a patient;
performing first biopsies at one or more locations in the prostate;
mapping the locations with the first image and storing the image and locations as a patient record;
at a time subsequent to obtaining the first image, obtaining a second image of the prostate of the patient;
registering the first and second images such that the locations of the first biopsies are overlaid onto the second image.

25. The method of claim 24, further comprising:
based on the locations of the first biopsies on the second image, identifying locations for second biopsies.

26. The method of claim 25, further comprising:
obtaining biopsy samples from the locations for second biopsies.

27. The method of claim 24, wherein the registering is performed in real time.
